(19) United States
(12) Patent Application Publication
(10) Pub. No.: US 2006/0093595 A1
(43) Pub. Date: May 4, 2006

(54) Methods, compositions and kits for treating damage to excitable tissue

(76) Inventor: Mahesh Jayachandra, Karnataka (IN)

Correspondence Address: MERCHANT & GOULD PC, P.O. BOX 2903, MINNEAPOLIS, MN, 55402-0903, US

(21) Appl. No.: 10/520,089

(22) PCT Filed: Jul. 2, 2003
(86) PCT No.: PCT/US03/20754

§ 371 Date: Oct. 4, 2005

Related U.S. Application Data
(60) Provisional application No. 60/393,977, filed on Jul. 5, 2002.

(51) Int. Cl. A61K 38/54; A61K 31/573; A61K 31/24; A61K 38/04

(52) U.S. Cl. 424/94.2; 514/19; 514/171; 514/537

(57) ABSTRACT

Drug cocktails including protease inhibitors, sodium channel blockers and corticosteroids are described. The use of these drug cocktails in treatment of damage due to injury to excitable tissue is discussed. The use of the drug cocktails in the treatment or prevention of degenerative conditions of the central nervous system and cardiovascular system are also discussed. Kits, and compositions including protease inhibitors, sodium channel blockers and corticosteroids are also described.

This application is being filed as a PCT International Patent Application in the name of Tetragrammaton Inc., a U.S. national corporation and resident, (Applicant for all countries except US) and Mahesh Jayachandra, a U.S. resident and IN citizen (Applicant for US only), on 2 Jul. 2003, designating all countries and claiming priority to U.S. Ser. No. 60/393,977 filed on 5 Jul. 2002.

### FIELD OF THE INVENTION

The present invention relates to methods, kits, and compositions useful for treating injury to excitable tissue. The invention also relates to compositions comprising a sodium channel blocker, a protease inhibitor, and a corticosteroid.

### BACKGROUND OF THE INVENTION

Injuries to excitable tissue provide great costs to society and are a source of discomfort to those affected by such injuries. There is a need to develop therapies, in addition to those currently available, to treat damage or disease caused by injury to excitable tissues.

### SUMMARY OF THE INVENTION

The present invention provides methods, kits and compositions for treating or preventing damage due to injury to excitable tissue.

In one embodiment the invention provides a method for treating damage due to an injury to an excitable tissue. The method includes determining whether a subject has suffered an injury to an excitable tissue and administering to the subject therapeutically effective amounts of compounds, wherein the compounds include a sodium channel blocker, a protease inhibitor, and a corticosteroid. In some embodiments wherein the excitable tissue is cardiac tissue, the sodium channel blocker is optional.

In another embodiment, the invention provides a composition. The composition can include a sodium channel blocker, a protease inhibitor, a corticosteroid, or combinations thereof.

In yet another embodiment, the invention provides a kit. The kit can include containers comprising a sodium channel blocker, a protease inhibitor, and a corticosteroid. The containers can be physically coded to allow a user to identify the contents of the container.

### DETAILED DESCRIPTION OF THE INVENTION

Terms and Definitions

All scientific and technical terms used in this application have meanings commonly used in the art unless otherwise specified. As used in this application, the following words or phrases have the following meanings, unless otherwise indicated:

As used herein, “excitable tissue” means tissue that contains excitable cells. Excitable cells are cells that respond actively to an electric stimulus and have an electrical charge differential across their cellular membranes. Excitable cells are generally capable of undergoing an action potential. Such cells typically express channels, such as voltage-gated, ligand-gated, and stretch channels, which allow flow of ions (potassium, sodium, calcium, chloride, etc.) across the membrane. Excitable tissue includes nervous tissue and muscle tissue, including cardiovascular tissue. Nervous tissue includes tissue of the peripheral nervous system and central nervous system. Central nervous tissue includes the brain and spinal cord. Cardiovascular tissue includes cells of the heart and associated nerves.

As used herein, “injury to excitable tissue” means that an event has occurred which results in damage to the tissue. The event can be a physical force, chemical or biological. Damage means any alteration in the state of the tissue or of some of its cells, interrupting or disturbing the performance of the vital functions thereof, as a direct or indirect result of an injury to excitable tissue. Damage can occur at the time of the event or can occur, for example, seconds, minutes, hours, weeks, months or years, after the event.

“Ischemic damage” refers to that damage which occurs as a result of ischemia which is defined as a low oxygen state usually due to obstruction of the arterial blood supply or inadequate blood flow leading to hypoxia in the tissue.

As used herein, a “subject” is an organism that has suffered from, is suspected as having suffered from, or is likely to suffer from an injury to an excitable tissue. The subject can be an animal. The animal subjects can be mammals. “Mammals” means any class of higher vertebrates that nourish their young with milk secreted by mammary glands. Mammals include humans, rabbits, mice, monkeys, horses, cows, elephants, giraffes, and the like.

As used herein, “determining whether a subject has suffered an injury to an excitable tissue” means the use of any diagnostic process which would reveal the presence of damage to excitable tissue. If the subject is a human, a physician could perform the diagnosis. If a conclusive determination cannot be made, yet the subject is suspected of having suffered an injury to an excitable tissue, treatment according to the invention would be appropriate.

“Treat,” “treating,” or “treatment” as used herein means to inhibit or block at least one symptom that characterizes a condition associated with injury to an excitable tissue in a subject threatened by, or afflicted with, the condition and includes: (i) preventing the damage from occurring to an excitable tissue of the subject which may have suffered an injury to an excitable tissue, but has not yet been diagnosed as having suffered the injury; (ii) inhibiting a condition associated with injury to an excitable tissue, i.e., arresting its development; or (iii) relieving the condition, i.e., causing regression of the disease. The term “inhibit” means to reduce by a measurable amount, or to prevent entirely. The term “preventing” means to keep that which is expected, probable, or possible from happening, existing, or causing consequences.

“Pharmaceutically acceptable” refers to that which is useful in preparing a pharmaceutical composition that is generally safe, and non-toxic, and includes that which is acceptable for veterinary use as well as human pharmaceutical use.

“Pharmaceutically acceptable salts” refers to those salts which are pharmaceutically acceptable, as defined above, and which possess and retain the desired pharmacological activity of the specific compound.

“Pharmaceutically acceptable carrier” refers to those carriers which are pharmaceutically acceptable, as defined above, and any material which, when combined with a biologically active compound, allows the compound to retain biological activity.

The term “therapeutically effective amount” refers to that amount which is sufficient to effect treatment, as defined above, when administered to a subject in need of such treatment. The therapeutically effective amount will vary depending on the subject and disease state being treated, the severity of the affliction and the manner of administration, and may be determined routinely by one of ordinary skill in the art.

Excitable Tissue

The invention provides methods, compositions and kits for treating damage due to injury to excitable tissue. In one embodiment, the invention provides a method for treating damage due to an injury to cardiovascular tissue, including the heart and associated nerves. In another embodiment, the invention provides a method for treating damage due to tissue of the nervous system. In still another embodiment, the method can be used to treat damage due to injury of the spinal cord or brain, including stroke.

The invention provides methods, kits, and compositions for treating damage due to initial conduction block and secondary degeneration following an injury to an excitable tissue. The initial conduction block that the invention can treat typically occurs within several hundred milliseconds and can last several seconds or longer before initial recovery is observed. The invention can treat disruption of homeostatic concentrations of sodium and potassium that may result from the initial conduction block. The secondary degeneration that the invention can treat can occur seconds, days, months, or years after the injury.

Spinal Cord Injury

The invention provides methods, compositions, and kits for treating damage due to spinal cord injury. The method includes administration of one or more sodium channel blockers, one or more protease inhibitors, and one or more corticosteroid. The compounds can be administered systemically or at the site of the injury.

In one embodiment the invention provides a method to treat interference with conduction in sensory and motor circuits resulting from spinal cord injury. In another embodiment, the invention provides a method for reducing local damage to segmental circuits, dorsal roots, ventral roots, and combinations thereof.

The method of the invention can be used to treat spinal cord injury associated biological, chemical or physical insults. Physical insults include hypoimpact and hyperimpact. Hypoimpact is an impact due to a physical force that typically leads to recovery without treatment. In this respect, the method of the invention can be used, for example, to hasten the recovery. Hyperimpact is an impact due to a physical force that typically produces a complete transverse myelopathy. Transverse refers to dysfunction at a particular level across the spinal cord, which can manifest as altered function below the particular level, while normal function is typically retained above the particular level. Myelopathy refers to spinal cord dysfunction of any cause. Processes that cause myelopathy include transverse myelitis, trauma, arthritis/bony malformation of the vertebrae which protect the spinal cord, vascular malformation or ischemia, and vertebral fracture from osteoporosis infection or malignancy. Myelopathy can also be caused by a process called syrinx, which refers to an enlarged cyst within the spinal cord. Accordingly, in one embodiment, the invention provides a method to prevent complete transverse myelopathy. The method of the invention is also useful for the treatment of partial myelopathy. The method can be used to treat myelopathy due to any cause. In one embodiment, the method is used to treat myelopathy due to trauma.

The method of the invention can be used as adjunct therapy to other useful techniques for treating damage due to spinal cord injury. For example, in one embodiment, the method of the invention includes local decompression of the spinal cord in addition to administration of one or more sodium channel blockers, one or more protease inhibitors, and one or more corticosteroids.

In one embodiment for treating spinal cord injury, the invention provides a method wherein a protease inhibitor is administered locally or systemically, a sodium channel blocker is administered locally, and a corticosteroid is administered systemically. The protease inhibitor (e.g., leupeptin) can be locally (e.g., infusion) administered at a concentration of about 200 μg/ml or more or can be administered systemically (e.g., intramuscularly or intraveneously) administered at a concentration of about 40 mg/kg. Preferably, the protease inhibitor is administered locally. The sodium channel blocker (e.g., procaine) can be administered locally at doses known to be effective for spinal anaesthesia. The corticosteroid (e.g., methylprednisone) can be administered systemically at a concentration of about 30 to about 50 mg/kg.

Brain Injury

The invention provides methods, compositions, and kits for treating damage due to brain injury. The method of the invention includes administration of one or more sodium channel blockers, one or more protease inhibitors, and one or more corticosteroid.

The invention is useful for treatment of brain injury due to physical force, chemical reaction, or of biological origin. For example, the invention is useful for treatment of damage from brain injury resulting from a physical insult or trauma to the head, from ischemia and anoxia, and from infection. In one embodiment, the invention is used to treat damage from brain injury due to physical trauma to the head, resulting in a penetrating head injury, skull fracture, concussion, and the like.

The invention can be used to treat damage directly or indirectly due to brain injury. For example, the invention can be used to treat brain damage due to cerebral edema; bruising; swelling; brain shift; brain swelling; cerebral ischemia; cerebrospinal fluid (CSF) leak; elevated intracranial pressure; hematomas, including intracranial, epidural, and subdural hematomas; contusions, including cerebral lacerations; hemorrhage; diffuse axonal injury or shearing injury; infection, including meningitis, epidural abscess, subdural empyemas, and brain abscess; epilepsy; cranial nerve deficits; pseudoaneurysms; arteriovenous fistulas; hydrocephalus; and the like.

The invention can be used to treat damage and clinical symptoms due to brain injury which occurs almost instantly, or which develops hours, days, weeks, months, or years after the trauma Symptoms associated with brain injury that the invention can treat include memory loss, headaches, sleep disorders, fatigue, anger, depression, seizures, and the like.

The clinical condition associated with the brain injury depends mainly on the mechanism and anatomical location of the lesion(s). The invention can be useful for treating clinical conditions associated with brain injury regardless of the mechanism and anatomical location of the lesion(s).

The method of the invention can be used as adjunct therapy to other useful techniques for treating damage due to brain injury. For example, in one embodiment, the method of the invention includes local decompression of the brain in addition to administration of one or more sodium channel blockers, one or more protease inhibitors, and one or more corticosteroids.

In one embodiment for treating brain injury, the invention provides a method wherein a protease inhibitor is administered locally or systemically, a sodium channel blocker is administered locally, and a corticosteroid is administered systemically. The protease inhibitor (e.g., leupeptin) can be locally (e.g., infusion) administered at a concentration of about 200 μg/ml or more or can be administered systemically (e.g., intramuscularly or intraveneously) administered at a concentration of about 40 mg/kg. Preferably, the protease inhibitor is administered locally. The sodium channel blocker (e.g., procaine or lidocaine) can be administered locally at doses known to be effective for local anaesthesia (e.g., about 4 to about 5 mg/kg). The corticosteroid (e.g., methylprednisone) can be administered systemically at a concentration of about 30 to about 50 mg/kg.

Stroke

The invention can be of benefit to a subject that has suffered from a stroke. The method of the invention includes administration of one or more sodium channel blockers, one or more protease inhibitors, and one or more corticosteroid.

The invention can be useful for treating the symptoms, conditions, or effects of a stroke. Stroke is characterized by the sudden loss of circulation to an area of the brain, resulting in a corresponding loss of neurologic function. The invention is useful for treating both ischemic and hemorrhagic stroke. Ischemic strokes can be characterized as the narrowing or clogging of a blood vessel that restricts blood flow such that enough blood cannot get through to keep the brain cells alive as a result of plaques, small blood clots, or emboli, and the like. With a hemorrhagic stroke, a blood vessel in the brain actually bursts or leaks

The invention is useful for treating the common symptoms of stroke including abrupt onset of hemiparesis, monoparesis, or quadriparesis; monocular or binocular visual loss; visual field deficits; diplopia; dysarthria; ataxia; vertigo; aphasia; or sudden decrease in the level of consciousness. The invention is also useful for treating complications seen particularly in hemorrhagic stroke such as the damage done to brain cells by the blood in the brain as it decomposes or the build-up of pressure in the brain as a result of blood leaking out.

The invention is useful for treating ischemic cascades and their effects that can be triggered by the loss of perfusion to a portion of the brain associated with a stroke, including the treatment of irreversible central infarction and the surrounding potentially reversible ischemic penumbra. On the cellular level, the invention can treat damage to neurons caused by excessive calcium influx following stroke. For example, the invention can treat the undesired effects of release of a number of neurotransmitters, including large quantities of glutamate. Such undesired effects include activation of N-methyl-D-aspartate (NMDA) and other excitatory receptors on other neurons, which can lead to depolarization, causing further calcium influx, further glutamate release, and local amplification of the initial ischemic insult. The invention can also inhibit activation various degradative enzymes, such as proteases, resulting from calcium influx. By inhibiting activation of proteases, the invention can inhibit the destruction of the cell membrane and other essential cellular structures. The invention can also treat damage due to free radicals, arachidonic acid, and nitric oxide generated by ischemica cascades, which could lead to further neuronal damage. The invention can also inhibit gene activation or the effects of genes activation due to ischemic cascades, which genes can lead to the formation of cytokines and other factors that in turn cause further inflammation and microcirculatory compromise.

The method of the invention can be used as adjunct therapy to other useful techniques for treating damage due to stroke. For example, in one embodiment, the method of the invention includes local decompression and/or reperfusion of the affected area in addition to administration of one or more sodium channel blockers, one or more protease inhibitors, and one or more corticosteroids.

In one embodiment for treating stroke, the invention provides a method wherein a protease inhibitor is administered locally or systemically, a sodium channel blocker is administered locally, and a corticosteroid is administered systemically. The protease inhibitor (e.g., leupeptin) can be locally (e.g., infusion) administered at a concentration of about 200 μg/ml or more or can be administered systemically (e.g., intramuscularly or intraveneously) administered at a concentration of about 40 mg/kg. Preferably, the protease inhibitor is administered locally. The sodium channel blocker (e.g., procaine or lidocaine) can be administered locally at doses known to be effective for local anaesthesia (e.g., about 4 to about 5 mg/kg). The corticosteroid (e.g., methylprednisone) can be administered systemically at a concentration of about 30 to about 50 mg/kg.

Myocardial Infarction

The invention can be of benefit to a subject that has suffered from a myocardial infarction. The method of the invention includes administration of one or more protease inhibitors, one or more corticosteroid, and optionally one or more sodium channel blocker.

The invention can be useful for treating myocardial infarction regardless of its etiology. For example, the invention can treat the rapid development of myocardial necrosis typically due to a critical imbalance between the oxygen supply and demand of the myocardium. The invention can also treat partial or complete occlusion of the vessel and subsequent myocardial ischemia due to, for example platelet aggregation, thrombus formation, fibrin accumulation, hemorrhage into the plaque, and varying degrees of vasospasm resulting from, for example, plaque rupture. The invention can also treat ventricular hypertrophy, hypoxia due to carbon monoxide poisoning or acute pulmonary disorders, emboli to coronary arteries, coronary artery vasospasm, arteritis, coronary anomalies, or myocardial infarction caused by the use of drugs such as cocaine, amphetamines, and ephedrine.

The invention can treat electrical instability of the remaining heart muscle tissue resulting from necrosis of the heart muscle associated with myocardial infarction. The invention can be useful for reducing the size in terms of area or volume of the necrosis. The invention can also be used to reduce or prevent electrical instability associated with necrosis. For example, the invention can prevent ventricular fibrillation (chaotic electrical disturbance), arrhythmias, conduction defects, and the like. The invention can be used to reduce or prevent complications associated with myocardial infarction, including sudden death; extension of infarction or re-infarction; congestive heart failure (pulmonary edema); cardiogenic shock; pericarditis; mural thrombosis, including embolization, myocardial wall rupture and tamponade; papillary muscle rupture, including valvular insufficiency; ventricular aneurysm formation; and the like.

The method of the invention can be used as adjunct therapy to other useful techniques for treating damage due to myocardial infarction. For example, in one embodiment, the method of the invention includes local decompression and/or reperfusion of the area of the infarct in addition to administration of one or more protease inhibitors, and one or more corticosteroids, and optionally one or more sodium channel blockers.

In one embodiment for treating myocardial infarction, the invention provides a method wherein a protease inhibitor is administered intravenously, a sodium channel blocker is administered intravenously, and a corticosteroid is administered intraveneously or subcutaneously. The protease inhibitor (e.g., leupeptin) can be administered in the jugular vein at a concentration of about 10 to about 40 mg/ml. The sodium channel blocker (e.g., lidocaine) can be administered locally at doses known to be currently clinically effective (e.g., drip at about 10 to about 50 mcg/kg/min). The corticosteroid (e.g., methylprednisone or hydrocortisone hemisuccinate) can be administered at a concentration of about 30 to about 50 mg/kg.

Sodium Channel Blockers

The invention provides methods, compositions and kits comprising sodium channel blockers. Ion channels include voltage-gated, ligand-gated, mechano/stretch-activated channels. Ion channels that allow the flow of sodium across the cellular membrane are classified as sodium channels. Compounds that bind to sodium channels or associated molecules and inhibit the flow of sodium through sodium channels are sodium channel blockers. Sodium channel blockers useful for the invention include those compounds that block the flow of sodium through sodium channels. In one embodiment, sodium channel blockers are those compounds that inhibit sodium flow (current) through voltage-gated sodium channels.

One or more sodium channel blockers can be included in the methods, compositions, and kits according to the invention. Suitable sodium channel blockers are known and include quinidine, procaine, disopyramide, lidocaine, tocainide, mexiletine, phenytoin, flecainide, encanide, propafenone, and amiodarone. In one embodiment, the sodium channel blocker is procaine.

Sodium channel blockers included in the compositions and kits of the invention are useful for treating damage due to injury to an excitable tissue. Injury to an excitable tissue typically results in an initial conduction block which can result within about 300 ms of the injury event. The initial conduction block is typically associated with a rapid increase in the extracellular concentration of potassium. It is believed that the increase in extracellular potassium concentration is accompanied by flow of sodium ions into the cell. Recovery of conductance is observed as intracellular and extracellular concentrations of sodium and potassium return to homeostatic levels. While not intended to limit the scope of the invention, it is believed that sodium channel blockers act to treat damage due to injury to excitable tissue by inhibiting the initial conduction block caused by prolonged sodium ion influx.

In one embodiment, the concentration of sodium channel blocker administered to treat damage due to injury is a concentration that can block the monophasic response. The monophasic response generally refers to a recordable electric phenomenom where one of the responses of a biphasic response is inhibited. Recordings made with two electrodes placed on a nerve trunk or tract composed of many neuronal axons are typically biphasic because the activity sensed by each electrode at slightly different times are approximate mirror images of opposite polarity. When the nerve or tract under the distal electrode is ligated or crushed, only one of these mirror images is recorded, yielding the monophasic response. The response is monophasic because the reference electrode is placed in the killed-end zone; that is, the ligated or crushed zone. Recording a monophasic response can be advantageous because cancellation effects of the mirror responses are avoided. Techniques for recording the monophasic response are known and are generally capable of detecting populations of axons with different conduction velocities. By way of example, monophasic responses can be recorded, for example, from a ligated posterior tibial nerve while monitoring antidromic conduction in the sensory dorsal columns.

Protease Inhibitors

The invention provides methods, compositions and kits comprising protease inhibitors. Proteases are enzymes that catalyze reactions that degrade or fragment proteins. All cells contain a number of proteases. As long as the cells are not damaged, the proteases do not result in general digestion of cellular components. However, once cells are damaged, proteases begin to act on cellular proteins unspecifically. Unspecific action of proteases can have damaging, including necrotic and apoptotic, effects on cells and tissues. Protease inhibitors are compounds that inhibit the catalytic activity of proteases.

Protease inhibitors useful for the invention include specific and non-specific protease inhibitors. Proteases include endoproteases and exoproteases. Endoproteases include serine, cysteine, aspartic and metallo-proteases. Within each of these classes of proteases, there are many specific proteases. By way of example, cysteine proteases include papain, calpain, cathepsin B, cathepsin L. Specific protease inhibitors are inhibitors that inhibit a specific protease. Non-specific protease inhibitors are those compounds that inhibit the catalytic activity of one or more specific protease. A good reference is Proteolytic Enzymes. Eds Robert J. Beynon and Judith S. Bond. Published by IRL Press at Oxford University Press, 1989. ISBN 0-19-963059-3 Pbk.

One or more protease inhibitor can be included in the methods, compositions, and kits according to the invention. In a preferred embodiment, the method, composition, or kit of the invention includes a non-specific protease or a cocktail of specific and/or non-specific protease inhibitors. Protease inhibitors and cocktails of protease inhibitors are available from commercial sources such as Sigma-Aldrich and Merck.

Examples of suitable non-specific proteases include aprotinin, which inhibits serine proteases including trypsin, chymotrypsin, plasmin, trypsinogen, urokinase, kallikrein, and human leukocyte; leupeptin which inhibits papain, calpain, trypsin, and cathepsin B; 4-(2-aminoethyl)benzenesulfonyl fluoride (AEBSF) which inhibits serine proteases including trypsin and chymotrypsin; and bestatin which inhibits aminopeptidases including leucine aminopeptidase and alanyl aminopeptidase. Other known peptidases include phenylmethylsulfonylfluoride (PMSF), EDTA, benzamidine, pepstatin, trans-epoxysuccinyl-L-leucylamido(4-guanidino)butane (E-64), N-Tosyl-L-lysylchlormethane (TLCK), N-Tosyl-L-phenylalanylchloromethane (TPCK), p-Aminobenzamidine (pABA), o-Phenanthroline (o-Phen), antipain, chymostatin, elatstatin, soybean trypsin inhibitor, IAA, cathepsin D, pepsin, rennin, VdLPFFVdL, 1,10-phenanthroline, phosphoroamidon, amastatin, bestatin, diportin A, diportin B, soybean trypsin inhibitor, EGTA and DFP, 6-amino-capronate, hirudine, trasylol, phosphoramidon, TIMP, cm-phe-leu, cbz-gly-gly-NH₂, cbz-gly-phe-NH₂, cbz-ala-phe, cbz-tyr-tyr, and 2-macroglobulin. In one embodiment the method, composition, or kit of the invention includes a leupeptin.

Useful protease inhibitors for the invention include those compounds that inhibit proteases associated with damage to excitable tissue following injury. One protease that has been implicated in damage due to injury to an excitable tissue is calpain. Accordingly, protease inhibitors useful for the invention include those compounds that inhibit calpain activity.

Many protease inhibitors are temperature sensitive and should be kept cold prior to administration. The temperatures at which the proteases should be stored are known. Instructions for storage for commercially available protease inhibitors will typically be provided by the manufacturer. Preferably, temperature sensitive protease inhibitors are administered as an ice-cold perfusate at or near the site of the injury.

Additionally, it is often desirable to dissolve protease inhibitors in dimethylsulfoxide (DMSO). The final concentration of DMSO concentration is preferably less than 0.5% by weight.

Corticosteroids

The invention provides methods, compositions and kits comprising one or more corticosteroid. Corticosteroids are naturally occurring steroid hormones produced in the outer layer of the adrenal glands and refer to a number of synthetic derivatives that have properties similar to those of the natural hormones. Examples of corticosteroids suitable for the invention include dexamethasone, triamcinolone acetonide, fluticasone propionate, cortisone, budesonide, methyl prednisone, mometasone furoate, and hydrocortisone hemisuccinate. In one embodiment, at least one of the corticosteroids is methyl prednisone or hydrocortisone hemisuccinate.

Corticosteroids used with the present invention should be used at doses capable of stabilizing membrane action and inhibiting inflammation. These doses can be readily determined by one of skill in the art.

Kit

The present invention provides a kit including a sodium channel blocker, a protease inhibitor, and a corticostroid. In one embodiment, the kit can further include one or more containers capable of separately storing each of the respective compounds. In another embodiment, one or more of the compounds can be stored in a given container. The containers can be physically coded, for example, by color to allow for easy identification of the content of the container.

In one embodiment, the kit of the invention is designed to facilitate mixing of the compounds in an efficient manner. The kit can contain a sodium channel blocker, a protease inhibitor, and a corticostroid, each in dosage form. In another embodiment, the kit comprises a sodium channel blocker, a protease inhibitor, and a corticostroid in a single dosage form.

The kit can include instructions for mixing the compounds at a desired dose prior to administration, if mixing is desired. The kit can also include instructions regarding the various dosage ranges at which the compounds should be administered and how to determine these dosages.

Administration

The invention provides a method for administering compounds to a subject in need thereof, the compounds comprising a sodium channel blocker, a protease inhibitor, and a corticosteroid. The compounds can be combined in a single composition and administered as a single dosage unit or can be administered separately in multiple dosage units. The compounds can be administered locally or systemically. For injuries associated with nervous tissue, the protease inhibitor and the sodium channel blocker are preferably administered locally, at or near the site of the injury. Local infusion (perfusion) of the compounds will likely result in beneficial decompression of the injured tissue. For injuries associated with cardiac tissue, the protease inhibitor is preferably administered into the internal jugular vein. Preferably, the corticosteroids are administered systemically.

The compounds should be administered as soon as possible after the injury. Preferably, the compounds are administered within 24 hours of the injury. More preferably, the compounds are administered within 8 hours of the injury. Even more preferably, the compounds are administered within 4 hours of the injury. Most preferably, the compounds are administered within 2 hours of the injury.

The compounds useful in the present invention can be formulated as pharmaceutical compositions and administered to a mammalian subject, such as a human patient or a domestic, farm, or laboratory animal, and like animals, in a variety of forms adapted to the chosen route of administration. The mode of administration is left to the discretion of the practitioner and can include but is not limited to oral, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin.

In one embodiment, the compounds are administered as a perfusate by infusion. The compounds can be dissolved or suspended in a pharmaceutically acceptable solution, such as phosphate buffered solution or Ringer's solution, and administered by perfusion. In one embodiment, the compounds are administered as an ice-cold perfusate. When administered as a perfusate, the compounds are preferably introduced at or near the site of the injury.

The compounds may also be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound.

The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the compounds may be incorporated into sustained-release preparations and devices.

The compounds may also be administered intravenously or intraperitoneally by infusion or injection. Solutions of the compounds or their salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compound may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, hydroxyalkyls or glycols or water-alcohol/glycol blends, in which the present compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

The compounds may also be administered transdermally through the use of a transdermal patch containing the active compound and a carrier that is mainly inert to the active compound, is generally non-toxic to the skin, and allows delivery of the agent for systemic adsorption into the blood stream via the skin. The carrier may take any number of forms such as creams and ointments, pastes, gels, and occlusive devices. The creams and ointments may be viscous liquid or semisolid emulsions of either the oil-in-water or the water-in-oil type. Pastes comprised of absorptive powders dispersed in petroleum or hydrophilic petroleum containing the active ingredient may also be suitable. A variety of occlusive devices may be used to release the active ingredient into the blood stream such as a semipermeable membrane covering a reservoir containing the active ingredient with or without carrier, or a matrix containing the active ingredient. Other occlusive devices are known in the literature.

For intranasal administration the compounds may be formulated as compositions useful for nasal administration such as drops, powders, or aerosols. For example, for nasal administration by inhalation, the compounds or compositions may be formulated into an aqueous or partially aqueous solution, which can then be utilized in the form of an aerosol. Formulations for nasal administration may also be solid and may contain excipients such as lactose or dextran, or may be aqueous or oily solutions for use in the form of nasal drops or metered spray. Nasal formulations may include dry powders suitable for conventional dry powder inhalers, liquid solutions or suspensions suitable for nebulization and propellant formulations suitable for use in metered dose inhalants.

Absorption of a compound administered intranasally can be enhanced by surfactant acids such as, for example, cholic acid, glycocholic acid, taurocholic acid, ehtocholic acid, chenodeoxyxholic acid, deoxyxholic acid, dehydrocholic acid, glycodeoxycholic acid, cyclodextrins, and the like in an amount in the range of about 0.2 to about 15 weight percent.

For intrabronchial inhalation or insufflation, compounds may be formulated as compositions useful for pulmonary administration such as atomized vapors, powders, or aerosols. Regardless of the form of the composition, administration by inhalation is designed to allow a compound to reach the lung, where the compound can typically be readily absorbed into the bloodstream. Pulmonary drug delivery can be achieved by different approaches, including liquid nebulizers, aerosol-based metered dose inhalers, and dry powder dispersion devices. Compounds or compositions may be formulated into an aqueous or partially aqueous solution, which can then be utilized in the form of an aerosol.

Aerosol-based metered dose inhalers may rely on the use of propellants carbon diaoxide, hydrofluorocarbons, or chlorofluorocarbons, including dichlorodifluoromethane, trichlorofluoromethane, dichlorotetrafluoroethane, and the like. Compounds or compositions can be conveniently delivered from an insufflator, nebulizer or a pressurized pack or other convenient means of delivering an aerosol spray. The dosage unit in the pressurized aerosol can be determined by providing a valve to deliver a metered amount.

Dry powder dispersion devices can also be use to delivering compounds that may be formulated as dry powders. The powder is preferably readily dispersible prior to inhalation by the subject to assure adequate distribution and systemic absorption. Compounds can be in the form of a dry powder composition, for example a powder mix of the compound and a suitable powder base such as lactose or starch. The powder composition can be presented in unit dosage form in, for example, capsules or cartridges or e.g. gelatin or blister packs from which the powder may be administered with the aid of an inhalator or insufflator.

For intranasal administration or administration by oral inhalation, compounds can be conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insulator may be formulated containing a powder mix of a compound of the invention and a suitable powder base such as lactose or starch.

Useful dosages of the compounds for use in humans can be determined by comparing activity in animal models. Methods for the extrapolation of effective dosages in mice, and other animals, to humans are known to the art; for example, see U.S. Pat. No. 4,938,949.

The amount of the compounds, or active salts or derivatives thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.1 to about 1000 mg/kg. Typically, the dose will be in the range of 0.5 to about 100 mg/kg.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Necessary modifications in dosage ranges can be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

The invention will now be illustrated by the following non-limiting Examples.

### EXAMPLES

The examples below involve a paradigm in anesthetized higher mammals, in particular cats and monkeys, which are designed to simulate as closely as possible human pathology. Indices of excitable tissue function will be primarily monitored, i.e., nerve conduction. Indirect indices, for example enzyme changes or imaging techniques, will be utilized to supplement the conduction data.

The examples provided below may be modified as one skilled in the art would understand to achieve similar results.

### Example 1

### Treatment of Spinal Cord Injury with Cocktail Including a Sodium Channel Blocker, a Protease Inhibitor, and a Corticosteroid

Overview

Briefly, the sensory and motor tracts in anaesthetized cat and monkey spinal cords are monitored using known techniques. Monophasic responses recorded from the Dorsal Columns (DC-sensory) and the Corticospinal Tracts (CT-motor) provide a rigorous way in which the effects of the trauma can be quantified. For the sensory system the spinal cord is stimulated above and below the trauma site and MR from the posterior tibial nerves is studied. The properties of the motor system are analyzed by recordings of the monophasic response to the same stimuli in the Pyramidal Tract and the Internal Capsule.

After trauma, one or more sodium channel blocker, one or more protease inhibitor, and one or more corticosteroid is locally infused at the trauma site in pharmacologically viable doses. The experiments include administering these drugs individually as well as in a cocktail fashion.

Example of Detailed Method

Cats are anesthetized initially with a 40 mg/kg I.P. injection of sodium pentobarbital, prior to venous (left cephalic vein) catheterization for smaller doses (10 to 15 mg). Monkeys are pre-anesthetized with ketamine (100 mg I.M.; gluteal site) prior to venous (left cephalic vein) catheterization for smaller doses (10 to 15 mg). The animals are then ventilated with a Harvard respirator after tracheal cannulation. The cats and monkeys are then inserted into a Kopf stereostatic head holder prior to further surgery. Adequacy of anesthesia is analyzed by papillary movement, absence of spontaneous head movements when gallamine levels fall, and recording of barbituate spindles from the exposed cerebral cortex or from the pyramidal or corticospinal tracts with ball electrodes.

During both stimulation and weight drop, gallamine triethiodide (initially 3 mg/kg I.V.) is used to relax the muscles of the animal while ventilated. In some animals, arterial pressure is monitered with a pressure transducer, connected to a cannula in the left femoral artery to check if gallamine causes a precipitous drop in pressure. EKG is monoitored with a thoracic, subcutaneous needle electrode and an extrathoracic reference electrode. Body temperature, monitored rectally, is maintained at 36-38° C. by a shielded, heating lamp. A wide left craniotomy is then performed to expose the sensory motor cortex. The dura is excised and the pial surface of the brain is protected from drying with a viscous mineral-oil mixture. Gel foam and bone wax will be used for hemostasis.

Two separate laminectomies are performed at C₁ and at T₅-T₁₀. In some experiments the lamina are spared at T₈-T₉ to facilitate undisturbed, caudal recordings from the lateral corticospinal tract (CT) at T₁₀. Some basiocciput is removed for stereotaxic access to the medullary pyramid.

In a given cat or monkey it is possible to do several experiments and keep the preparation alive for as long as two days by suitable I.V. alimenation (20-30 cc dextrose-saline of Mammalian Ringer/day)

A modified Allen weight drop technique is used in which a constant weight (about 47 gm steel cylinder/impact diameter=0.5 cm) is dropped through a hollow transparent tube oriented perpendicular to, and symmetrically above the spinal cord. After impact, the weight is manually retracted. Sufficient lateral bone is removed to allow the weight to fall freely on the dura. The weight drop is performed over an intact dura or incised dura, where the incision is extended over three to four vertebral segments, as weight drops on dura with small incisions tends to result herniation. The 47 gm weight is dropped from distances varying from about 0.5 cm to greater than 8 cm above the point of impact at the exposed spinal cord.

The right posterior tibial nerve is exposed deep to the Achilles tendon, ligated, cut, covered with Vaseline-mineral oil mixture, and then suspended in air. Bipolar silver electrodes, interpolar distance 5-8 cm, are used to record monophasic (“killed-end”) responses to antidromic dorsal column (DC) stimulation. If the amplitude of the responses decline with time, the nerve is removed and cleaned. To correct for changes at the recording site over time, a second ‘normalizing stimulus’ is applied distal to the trauma site at T₁₀. A control deafferenation experiment is performed to check for any motor contamination.

Recording is done from the lateral corticospinal tract (CT). Orthodromic direct population responses to primary motor cortical or internal capsular stimulation are obtained extracellularly with a tungsten electrode (125 μm in diameter and coated with Teflon to its tip). The electrode is inserted into the contralateral spinal cord lateral to the midline at T₁₀. Alternatively, antidromic direct population responses are recorded in the contralateral medullary pyramid to epidural, spinal cord stimulation above and below the trauma site.

A silver ball electrode is placed on the epidural surface over the dorsal lateral spinal cord to record changes in direct population responses as a function of distance from the lesion.

To stimulate the primary afferents antidromically in the dorsal column, bipolar silver wires, insulated at the tips, with Teflon, are placed on the pia over fasciculus gracilis at C₁-C₂ level or epidurally at T₅ and T₁₀. In each case, the stimulus polarity eliciting the earlier (usually lower threshold) response is used to ensure that the cathode lay closest to the recording electrodes. Thus the primary afferents are stimulated both above and below the level of the trauma, thereby preventing long term changes, e.g., at the site of the posterior tibial nerve recording, to be detected and the responses to stimulation above the lesion to be normalized.

The lateral CT fibers are stimulated antidromically above and below the trauma site. Besides driving the CT, motor cortex stimulation are likely useful in obtaining optimum recordings from the medullary pyramid. At the internal capsule, corticospinal fibers are also subcortically orthodromically activated with bipolar, adjoining nichrome wires (125 μm diameter; insulated to their tips with Formvar).

Rectangular pulses, isolated from ground, are used for electrical stimulation where the stimulus frequency will usually be set at about 1-2 Hz and the duration of the pulse being about 50-200 msec. Stimulus intensities, recorded in arbitrary units, are kept at a constant supramaximal level throughout the experiment. The stimulating current is also monitored with a Tektronix current probe (model P6016) whose sensitivity is increased about ten times by coiling ten turns of wire through the probe.

Single units are recorded extracellularly in M1 cortex using electro-pointed tungsten microelectrodes insulated with Formvar till their tips. The units are antidromically fired by supramaximal CT stimulation above and below the trauma site. Antidromic conduction can be shown by relatively invariant latency of discharge and high frequency following (1:1 or 1:2) in response to a short 1 KHz train. Latencies from above the site are normalized for distance separating the electrodes.

After a low lumbar laminectomy, the cauda equina is dissected under binocular vision. Individual roots are isolated, separated by glass probes, and mounted on a Perspex platform (5×3×1.5 mm) over which either a 7.5 gm or 15 gm brass rod will be dropped through a transparent plastic tube. In some experiments, the distant part of the root is cut and monophasic responses are recorded in the typical manner. In other experiments, where the distances involved are small, the posterial tibial nerve is recorded to avoid direct effect of trauma at the recording site or caudal spread of stimulating current. K⁺ depolarization of the cauda is performed by topically applying cotton pledget soaked in appropriate concentration of K⁺ in mammalian Ringer's solution.

In the above experiments, one or more sodium channel blocker, one or more protease inhibitor, and one or more corticosteroid is locally infused at the trauma site in pharmacologically viable doses after trauma. The experiments include administering these drugs individually as well as in a cocktail fashion.

At the end of the experiment, the animal is killed by I.V. standard euthanasia fluid (high KCl and pentoparbital).

The electrophysiological data is conventionally (resistance-capacity coupled) amplified and recorded on a multichannel analog, frequency modulated tape. The electrodes are connected to a cathode-follower head stage which will feed into a Tektronix FM 112 differential preamplifier (filter settings at 0.8 Hz and 1 or 10 KHz) and then into operational amplifiers and to various display and storage devices—a storage oscilloscope (Textronix 511A), an 8 channel FM tape recorder (Sanborn 2000), an audio amplifier, a computer of average transients with an X-Y plotter (Moseley 150) and an IBMPC-AT computer with a DT2801A data acquisition board, ASYSTANT PLUS software (@Macmillan) are used for data acquisition and analysis (sampling rate=10 KHZ; single scans or averages of 5-20) and SIGMA PLOT is used for graphs.

### Example 2

### Treatment of Mechanical Brain Injury and Stroke with Cocktail

### Including a Sodium Channel Blocker, a Protease Inhibitor, and a Corticosteroid

Overview

For mechanical brain injury and stroke experiments, the methods of monitoring brain function are similar. The method of damage to the brain differs.

Experimental indices initially focus on a sensory (auditory) and the motor system:
1. Tract function is monitored by recording the ‘D’ wave of the monophasic response in the pyramidal tract (motor) secondary to cortical electrical stimulation of the motor area (M1) by silver ball electrodes.
2. Intemeuronal function is monitored by recording the ‘I’ wave of the monophasic response in the pyramidal tract (motor) secondary to cortical electrical stimulation of the motor area (M1) by silver ball electrodes.
3. Columnar function is monitored in the auditory cortex by the current source density (CSD) response to binaural clicks. Here a linear array of 8-16 electrodes is used to straddle the cortical column and record from it as a unit. (Jayachandra M & P. Kumar, Soc. Neurosci Abstr. 823.5, San Diego, 2001). These provide:
   i. The intracortical auditory evoked responses: the general response of the auditory cortex.
   ii. The CSD response which reflects the post-synaptic activity
   iii. The multi-unit activity (MUA) which reflects neuronal activity.
      Methods of Damage to the Brain:
1. Stroke: Cerebral Artery Occlusion (e.g., carotids, middle cerebral, Circle of Willis) is performed both unilaterally as well as bilaterally in the anaesthetized cat or monkey. The level of trauma is quantified, e.g., time of occlusion of blood flow. Functional recovery is studied by systemic infusion of individual drugs as well as the whole cocktail.
2. Mechanical Brain Injury is caused by focused mechanical concussion of the brain prior to drug infusions. The level of trauma is controllable and functional recovery is studied in a similar fashion. Local applications of drugs are also tried in the mechanical brain trauma model.

### Example 3

### Treatment of Myocardial Infarction with Cocktail including a Sodium

### Channel Blocker, a Protease Inhibitor, and a Corticosteroid

Overview

Heart function is monitored by EKG, blood pressures, and cardiac enzymes by known procedures.

The coronary arteries (e.g., anterior descending coronary artery) are ligated to simulate a myocardial infarction. The level of trauma is standardized by the time of occlusion. The functional indices are monitored throughout. The drugs is systemically administered as a cocktail as well as individually to achieve functional recovery. Post-mortem histological analysis of the infarcted heart provides additional data regarding the therapeutic potential of these interventions. Additional imaging techniques may be used to supplement the findings.

We claim:

1. A method comprising:
determining whether a subject has suffered an injury to an excitable tissue; and
administering to the subject therapeutically effective amounts of compounds, wherein the compounds comprise a sodium channel blocker, a non-specific protease inhibitor, and a corticosteroid.

2. The method of claim 1, wherein the sodium channel blocker is procaine.

3. The method of claim 1, wherein the non-specific protease is leupeptin.

4. The method of claim 1, wherein the corticosteroid is selected from the group consisting of methylprednisone and hydrocortisone hemisuccinate.

5. The method of claim 1, wherein the corticosteroid is methylprednisone.

6. The method of claim 1, wherein the non-specific protease inhibitor is administered as an ice-cold perfusate.

7. The method of claim 1, wherein the compounds are administered as a single drug dose.

8. The method of claim 1, wherein the compounds are administered in separate drug dosages.

9. The method of claim 1, wherein the compounds are administered within eight hours of the injury to the excitable tissue.

10. The method of claim 9, wherein the compounds are administered within four hours of the injury to the excitable tissue.

11. The method of claim 10, wherein the compounds are administered within two hours of the injury to the excitable tissue.

12. The method of claim 1, wherein one or more of the compounds is administered systemically.

13. The methods of claim 1, wherein one or more of the compounds are administered by perfusion at or near the injured tissue.

14. The method of claim 1, wherein the injury is to the central nervous system.

15. The method of claim 14, wherein the injury is to the brain.

16. The method of claim 15, wherein the injury results from ischemic damage to the brain.

17. The method of claim 16, wherein the injury results in stroke.

18. The method of claim 14, wherein the injury is to the spinal cord.

19. The method of claim 1, wherein the injury is to the heart.

20. The method of claim 19, wherein injury is myocardial infarction.

21. The method of claim 1, further comprising decompressing the injured tissue.

22. The method of claim 1, further comprising reperfusing the injured tissue.

23. A composition comprising:
a sodium channel blocker;
a non-specific protease inhibitor;
a corticosteroid; and
a pharmaceutically acceptable carrier.

24. The composition of claim 23, wherein the composition comprises therapeutically effective amounts of the sodium channel clocker, the protease inhibitor, and the corticosteroid.

25. A kit comprising:
a sodium channel blocker;
a non-specific protease inhibitor; and
a corticosteroid.

25. The kit of claim 24, further comprising separate containers for storing the sodium channel blocker, the non-specific protease inhibitor, and the corticosteroid.

26. The kit of claim 25, wherein the containers are color coded to allow for easy identification of the contents of the containers.

27. The kit of claim 24, wherein the sodium channel blocker, the protease inhibitor, and the corticosteroid are each in dosage forms.

28. The kit of claim 27, wherein the sodium channel blocker, the protease inhibitor, and the corticosteroid are in a single dosage form.
